(19) United States
(12) Patent Application Publication
(10) Pub. No.: US 2015/0272727 A1
(43) Pub. Date: Oct. 1, 2015

(54) INTRAOCULAR LENS WITH COUPLING FEATURES

(71) Applicants: Mark Humayun, Glendale, CA (US); Charles DeBoer, Sierra Madre, CA (US); Yu-Chong Tai, Pasadena, CA (US); Sean Caffey, Pasadena, CA (US); Craig Alan Cable, II, Mission Viejo, CA (US)

(72) Inventors: Mark Humayun, Glendale, CA (US); Charles DeBoer, Sierra Madre, CA (US); Yu-Chong Tai, Pasadena, CA (US); Sean Caffey, Pasadena, CA (US); Craig Alan Cable, II, Mission Viejo, CA (US)

(21) Appl. No.: 14/670,686

(22) Filed: Mar. 27, 2015

Related U.S. Application Data
(60) Provisional application No. 61/971,230, filed on Mar. 27, 2014.

(51) Int. Cl. A61F 2/16

(52) U.S. Cl. CPC A61F 2/1635; A61F 2002/1682; A61F 2002/16901; A61F 2250/0009; A61F 2250/001; A61F 2250/0013; A61F 2250/0051; A61F 2250/0071; A61F 2250/0091

(57) ABSTRACT

Mechanical and/or adhesive coupling features are added to an intraocular lens during fabrication or thereafter, and provide beneficial mechanical coupling to the lens capsule. The coupling features may pass through or otherwise engage complementary coupling apertures in the crystalline lens capsule.

### CROSS-REFERENCE TO RELATED APPLICATIONS

This application claims priority to and the benefit of U.S. Provisional Application No. 61/971,230, filed on Mar. 27, 2014, the entire disclosure of which is hereby incorporated by reference.

### BACKGROUND

Intraocular lenses (IOLs) are used to replace the natural crystalline lens after cataract removal. First the cataract is surgically removed from the capsule of the crystalline lens. To implant an IOL, an incision is made in the cornea, followed by a capsulorhexis—i.e., removal of a portion of the lens capsule to provide surgical access to the natural lens. Most often, a central 5-6 mm capsulorhexis is made. The natural lens is removed using phacoemulsification in a process of fragmenting and aspirating the lens from the lens capsule. The IOL, in a folded conformation, is then injected into the lens capsule. The implanted IOL is filled with liquid to provide the appropriate amount of vision correction.

More recently, lasers have been used to make the main corneal incision and capsulorhexis, and to fragment the lens. With these technologies, more advanced shapes of capsulorhexis are possible. In addition, advanced features or holes can be made in the lens capsule.

Many types of IOLs have been approved for use and others are in development. The most common are monofocal IOLs, multifocal IOLs, toric IOLs, and accommodating IOLs. Monofocal IOLs, the most common intraocular lens implanted, are not adjustable; they provide vision at a specific focal plane. Multifocal IOLs were developed to provide simultaneous near and far focal points, giving the patient increased depth of visual field. Multifocal IOLs often provide near and far focusing percentages as a function of pupil size—that is, the amount of near focus (or the “near add,” which refers to the total lens power needed over a given distance prescription to allow for clear up-close vision) varies with the pupil diameter. Therefore, the location of the IOL relative to the pupil is critical for accurate functioning.

Tonic IOLs correct astigmatism in the cornea, and require a specific angular orientation after implantation to ensure proper optical functioning. In the extreme case, when the toric is 90° from its intended angular position, it increases rather than corrects the astigmatism. For this reason, toric IOLs contain a fiducial marker to indicate angular position. After implantation, the toric IOL is rotated into the correct location.

Accommodating intraocular lenses (AIOLs) focus in response to the eye's natural focusing muscle, the ciliary muscle. Therefore, just like the youthful natural lens, these lenses eliminate the need for reading glasses. AIOLs often require coupling to the ciliary muscles of the patient's eye or the lens capsular bag. Other AIOL designs monitor the pupillary diameter or tonus of the ciliary muscles, and based on this, they adjust—either passively by being acted upon by the muscles or actively as in the case of an electroactive lens.

In all scenarios the position of the IOL is critical for proper functioning. Tip, tilt, angular displacement or decentration reduce the IOL's optical performance, often to a severe extent in the case of toric, multifocal, and accommodating IOLs. Subsequent ocular procedures (e.g., treatment for posterior capsular opacification) can displace an otherwise properly implanted IOL and impair performance—for example, an additional opening in the lens capsule can lead to decentering of the IOL, and in some cases the IOL may even fall out of the lens capsule into the posterior chamber of the eye.

### SUMMARY

In various embodiments, the present invention relates to coupling features that are added to an IOL during fabrication or thereafter, and which provide beneficial mechanical coupling to the lens capsule. By “coupling” is meant a mechanical (including adhesive) interaction that promotes stability of the IOL by anchoring it within the lens capsule, thereby eliminating or opposing relative movement between the IOL and the lens capsule. Coupling features may, in various embodiments, be shaped to interact (e.g., mate) with specific apertures or recessed features, referred to as coupling apertures, on the lens capsule. The coupling apertures may be on any position along the lens capsule, and are typically created surgically. The coupling apertures and coupling features are desirably outside the central optical axis of the eye. In certain embodiments, they are maintained outside the central 2 mm of the IOL. In other embodiments, they are maintained outside the central 3 mm or 4.25 mm of the IOL.

Mating between coupling features on the IOL and coupling apertures in the lens capsule allow the IOL to be located, and remain, in a specific angular and concentric orientation to the lens capsule and the eye, and provide a mechanical connection between the lens capsule and the IOL. Complementary profiles between the coupling features and apertures may provide a locating function, allowing, e.g., for improved IOL centering with respect to the eye. In addition, angular positioning can be used to increase the optical performance of the IOL. As an example, a toric IOL performs best at a specific angular orientation. Accordingly, based on the desired IOL orientation for that patient and anatomical constraints in placing coupling apertures, one or more custom coupling features can be designed for the patient so as to align with one or more coupling apertures.

Coupling features also ensure that the IOL does not migrate over time, and that the IOL is centrally located. As an example, this is important for IOLs that are meant to interact with a patient's pupil. Multifocal IOLs often have varying amounts of near add based on pupil diameter. Therefore, misalignment with the pupil prevents the multifocal IOL from increasing the add appropriately with pupillary diameter changes. By aligning the multifocal IOL with the eye and pupil, proper functionality of the IOL is ensured and retained.

Coupling features also ensure that the IOL does not sublux (i.e., shift position) from either the anterior or posterior capsulorhexis in the lens capsule. Subluxing is higher risk after posterior capsulorhexis, which is used after PCO treatment or during IOL implantation in a pediatric case. For example, a posterior capsulorhexis is cut into the posterior lens capsule during pediatric implantation, or after the treatment for PCO. Coupling features ensure that the lens does not sublux or become dislocated from the lens capsule.

For accommodating IOLs, coupling features in the IOL can be used to transmit forces from the lens capsule to the IOL itself. In the case of an accommodating liquid-filled IOL, as described, for example, in U.S. Patent Publ. No. 2012/0303118 (the entire disclosure of which is hereby incorporated by reference), the lens capsule transmits a tangential and normal force to the IOL as it brings the IOL into far displacement. Coupling features may be employed to prevent slipping of the IOL against the lens capsule. These features ensure that the IOL is compressed and pulled tangentially as the lens capsule stretches, increasing the efficacy of the IOL and preventing unwanted bulging or edge deformities of the IOL relative to the edge of the capsulorhexis.

In other embodiments, the coupling feature is a valve on the lens. This is particularly useful with liquid-filled IOLs (see, e.g., U.S. Pat. No. 8,771,347). The valve may attach to the lens capsule, e.g., to an aperture in the lens capsule or by means of a portion on the valve that adheres to the lens capsule—e.g., by means of a series of micro or macro features, grooves, or hooks that cause it to stick to the capsule, or roughened areas of the valve into which the lens capsule tissue grows and adheres. Other features of the IOL, such as preexisting haptics, may be modified by added coupling features or other treatments to become points of attachment to complementary apertures in the lens capsule. In certain embodiments, the valve is used to increase stretching of the lens with the lens capsule. This can increase accommodation or make accommodation more repeatable for a liquid lens. In other embodiments, the valve is used prevent rotation.

Coupling features and coupling apertures may be integrated into an intermediate member containing profiles complementary to both the lens capsule and the IOL. The intermediate member or capsule coupler provides the benefits of the coupling interaction described above, and additionally transmits forces from the lens capsule to the IOL more efficiently. In the case of liquid-filled IOLs, the capsule coupler may be employed to reinforce the IOL and protect it from external forces that might otherwise rupture the IOL membrane.

Accordingly, in a first embodiment, the invention pertains to an intraocular lens comprising a polymeric lens body having a refractive power and configured for implantation to a patient's lens capsule, and one or more coupling features on the lens body for anchoring the lens body to the lens capsule. The coupling feature(s) may include a plurality of protrusions from a surface of the lens body. For example, the protrustions each may include a catch (e.g., a hook, ledge, circular protrusion, or coned feature) that latches over the lens capsule. Alternatively or in addition, the coupling feature(s) may include one or more adhesive patches that adhere the lens body to the lens capsule; for example, the adhesive may be a chemical adhesive and/or a mechanically featured adhesive.

In some embodiments, the coupling feature(s) are a surface treatment on the lens body promoting adhesion to the lens capsule. The coupling feature(s) may be outside a central optical axis of the lens—e.g., outside a central 2 mm or 3 mm of the lens.

In various embodiments, the lens further comprises an intermediate member with coupling features complementary to the lens body and the lens capsule. The intermediate member may be unitary or may comprise a plurality of segments. For example, the segments may be breakably joined to each other.

In another aspect, the invention pertains to a sensor coupling system. In various embodiments, the system comprises a polymeric lens body having a refractive power and configured for implantation to a patient's lens capsule; one or more coupling features on the lens body for anchoring the lens body to the lens capsule; a sensor for measuring a force exerted on the one or more coupling features; a signal interpreter for interpreting sensor measurements; and means, responsive to the signal interpreter, for altering the refractive power of the lens body based at least in part on measurements from the sensor.

The signal interpreter may be configured to determine a state of the patient's eye. In some embodiments, the means responsive to the signal interpreter comprises or consists of an actuator for altering a conformation of the lens body via the coupling features. Alternatively, the means responsive to the signal interpreter may comprise a pump for driving a fluid into and out of the lens body.

The term “substantially” or “approximately” means ±10% (e.g., by weight or by volume), and in some embodiments, ±5%. The term “consists essentially of” means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1A is a partial cutaway view depicting an IOL with coupling features implanted into a lens capsule with coupling apertures.

FIG. 1B is an enlarged sectional view of an implanted IOL, illustrating mechanical engagement of the coupling feature shown in FIG. 1A within a coupling aperture.

FIG. 1C is an enlarged sectional view of an implanted IOL having a coupling feature with an additional retention feature.

FIG. 2 is a partial cutaway view depicting a dual-optic intraocular lens with a coupling feature implanted into a lens capsule.

FIG. 3 is a partial cutaway view depicting an IOL that does not change focal length.

FIG. 4 depicts the configuration and operation of a representative sensor system employing an IOL coupler for determining the position of the ciliary muscle.

FIG. 5 is a sectional view of an embodiment in which an intermediate member intervenes between an IOL and the patient's lens capsule.

FIG. 6 is a sectional view showing an intermediate member just slightly larger than the capsulorhexis opening.

FIG. 7 is a sectional view of an embodiment in which a pair of intermediate members intervene between an IOL and the patient's lens capsule.

FIG. 8 is a perspective view of an embodiment in which four intermediate members intervene between an IOL and the patient's lens capsule.

### DETAILED DESCRIPTION

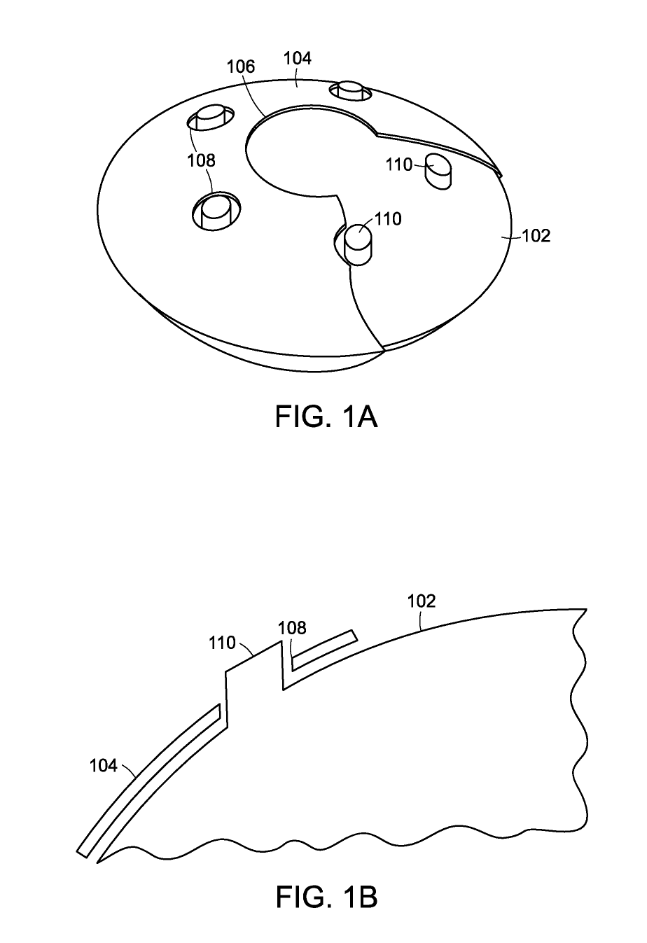

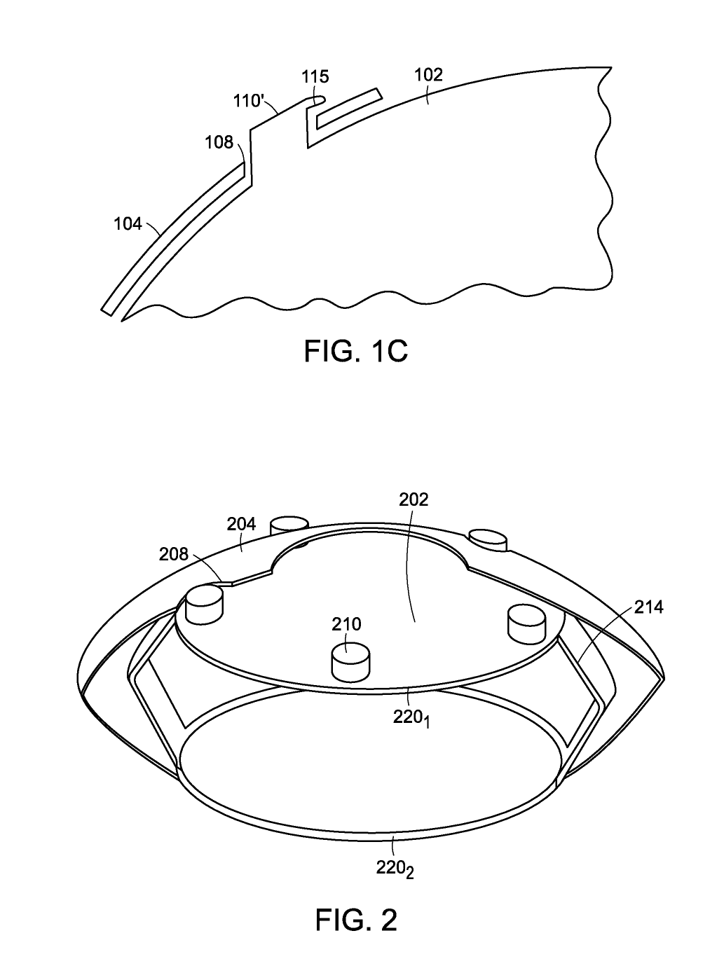

Refer first to FIG. 1A, which depicts an IOL 102 implanted into a lens capsule 104 of a patient. A capsulotomy (incision) 106 has been made in the lens capsule 104 (which has a section schematically cut away for illustrative purposes). Depending on the procedure, the capsulotomy 106 may be, for example, between 1 and 8 mm in diameter. It may be along the central optical axis of the eye, or decentered. In addition, one or more coupling apertures 108 are surgically cut into the lens capsule. The IOL has a series of raised coupling features 110 (posts in the illustrated embodiment) that mechanically engage the coupling apertures 108 in the lens capsule, thereby restraining movement of the IOL 102 relative to the lens capsule 104. In addition, the lens capsule 104 can effectively transmit force to the IOL 102 via the coupling features 110. For example, coupling can be used to transmit accommodation forces to the IOL 102 to cause a shape or dimensional change therein.

Coupling apertures in the lens capsule may be made manually, such as by using capsulorhexis forceps, cautery, or specific manual tools, or may be made optically using, for example, a Nd:YAG or femtosecond laser. In certain embodiments, the coupling apertures are made using computational analysis of the lens, the lens capsule, desired orientation, optical performance of the implanted lens, and/or the geometry or anatomy of the eye. When this is done, the predicted location of the lens can be modeled before the coupling apertures are made.

Coupling apertures may be made before the surgery, during the surgery, or following implantation. In one embodiment, the coupling apertures are made before implantation of the lens, and in some embodiments before removal of the natural lens. As an example, a femtosecond laser may be used to make coupling apertures in the lens capsule before lens removal. Then cataract surgery is performed and the IOL is implanted. In another example, a toric IOL is implanted and determined to be in the correct location—e.g., by using fiducial markers or through optical analysis (e.g., using an intraoperative autorefractor or aberrometer). Next, based on the location of the coupling features, coupling apertures are made in the lens capsule during or after the surgery. In certain embodiments, only when the coupling apertures are opened does the engagement between coupling features and coupling apertures occur. The coupling apertures may be made in the lens capsule following the initial surgery (e.g., six months to one year thereafter) to ensure correct placement after settling. In the case of a liquid-filled IOL, the IOL fill volume may be adjusted prior to opening the coupling apertures in the lens capsule. Stiction forces still maintain the IOL's relative position within the aperture prior to the coupling apertures being opened.

FIG. 1B shows a coupling feature 110 and its engagement with a coupling aperture 108 in greater detail. The illustrated coupling feature 110 does not extend perpendicularly from the curved surface of the IOL 102, but instead is angled so as to extend parallel to the patient's visual axis. This maximizes the distance of the coupling feature 110 from the central lens axis of the IOL 102. In FIG. 1C, the coupling feature 110′ has an additional retention feature 115, which latches over the lens capsule 104 prevents the coupling feature 110′ from slipping underneath the lens capsule should the IOL 102, for example, retract from the lens capsule 104.

It should be stressed that the retention feature 115 may take many shapes and forms to prevent the lens capsule from slipping over and becoming dislodged from the IOL 102. For example, hooks, ledges, circular protrusions, and/or coned portions may be used to prevent dislodgement of the IOL 102 while avoiding difficulty in passing the coupling features 110 through the coupling apertures 108. In other embodiments, the IOL 102 is twisted into place, and one or more retention features hook onto the surrounding lens capsule 104. In yet other embodiments, the coupling features 110 and/or the coupling apertures 108 stretch so that the coupling features are more securely retained within the coupling apertures. In all of these scenarios, the coupling features 110 restrain movement, migration, tilt, or rotation of the IOL 102. Under certain conditions, the coupling apertures 108 in the lens capsule 102 reduce in size over time (e.g., due to capsular phimosis) and become strongly adherent to the coupling features in the intraocular lens over time.

The coupling features may be used for locating and centering the IOL after implantation, e.g., in the case of astigmatism-correcting IOLs such as toric IOLs. More generally, this approach is useful for centering IOLs with haptics and IOLs without haptics. Examples of IOLs without haptics include certain inflatable IOLs and conformal-fitting IOLs.

In some embodiments, the coupling features are adhesive in addition to, or in lieu of, a mechanical protrustion. Examples of suitable chemical adhesives include pressure-sensitive adhesives, temperature-sensitive adhesives such as poly(N-isopropylacrylamide), adhesives that bond over a period of time, cyanoacrylates, silicones, silicone gels, and mechanically featured adhesives. Examples of mechanical featured adhesives include but are not limited to micropatterned hooks, grooves, and knobs, which mechanically attach to the lens capsule. Exemplary methods to manufacture these include surface treatment of the IOL and micropatterning of the IOL surface. Other ways of promoting adhesion include surface treatment to change the chemical properties of the IOL surface. Examples of chemistry-altering surface treatments include oxygen plasma treatment, ammonia treatment, and HDMS treatment. These can make the surface more hydrophilic or hydrophobic. In some embodiments, a molecular side chain is added to the IOL surface to beneficially alter surface properties. Surface treatment can be used promote stronger adhesion between the IOL and the lens capsule surface, or to prevent migration of lens epithelial cells in order to reduce posterior or anterior capsular opacification. In yet other embodiments, the surface is modified with a pharmacological group that prevents cell migration (e.g., is cytotoxic to lens epithelial cells, thereby preventing their migration) or differentiation. Such coupling features may not need a coupling aperture and may instead interact directly with the lens capsule.

In some implantation procedures, the coupling features may be passed through the coupling apertures before filling or full deployment of an IOL (e.g., in the case of a fluid-filled IOL, multi-optic IOL, or multi-part IOL). As an example, a fluid-filled IOL may be implanted partially or fully empty. The coupling features are initially passed through coupling apertures, and only then is the IOL filled. Alternatively, the coupling apertures may engage the coupling features during filling. In still other procedures, the coupling features of one part of a multi-part IOL are engaged with coupling apertures. Then the coupling features of additional portions of the multi-part IOL are engaged with corresponding coupling apertures. In the case of an expandable IOL, the coupling features and coupling apertures may be mated before, during, or after the IOL is expanded. As an example, a dual-optic IOL may be implanted into the lens capsule. After deployment, the coupling apertures and coupling features engage as the two optics are brought apart.

FIG. 2 depicts a dual-optic intraocular lens 202 implanted into a lens capsule 204. Once again, the coupling apertures 208 receive the coupling features 210. In addition, the dual-optic IOL 202 has a hinge 214 that couples the first optic 220₁ to the second optic 220₂. This ensures proper orientation of the dual-optic IOL 202 and also transmits force from the lens capsule 204 to the IOL 202 when the lens capsule 204 changes shape, e.g., during accommodation. Similar techniques can be used with single-optic vaulting IOLs, dual-optic IOLs that shift by a rotation of one optic relative to a second optic, etc.

In some procedures, the coupling apertures are located on the anterior portion of the lens capsule. However, they may be located on the posterior portion, the equatorial region, or any other region or combination of regions in the lens capsule. As an example, when located on the posterior of the lens capsule, the coupling apertures prevent the lens from either subluxing posterior or anterior, and prevent IOL dislocation or IOL movement. This can reduce complications from opening the posterior portion of the IOL, as in the case us Nd:YAG or femtosecond laser treatment of the posterior capsule for PCO. In addition, these locating apertures can be used to ensure proper placement of multifocal IOLs, or other IOLs that require centering and positional locating for optimal performance.

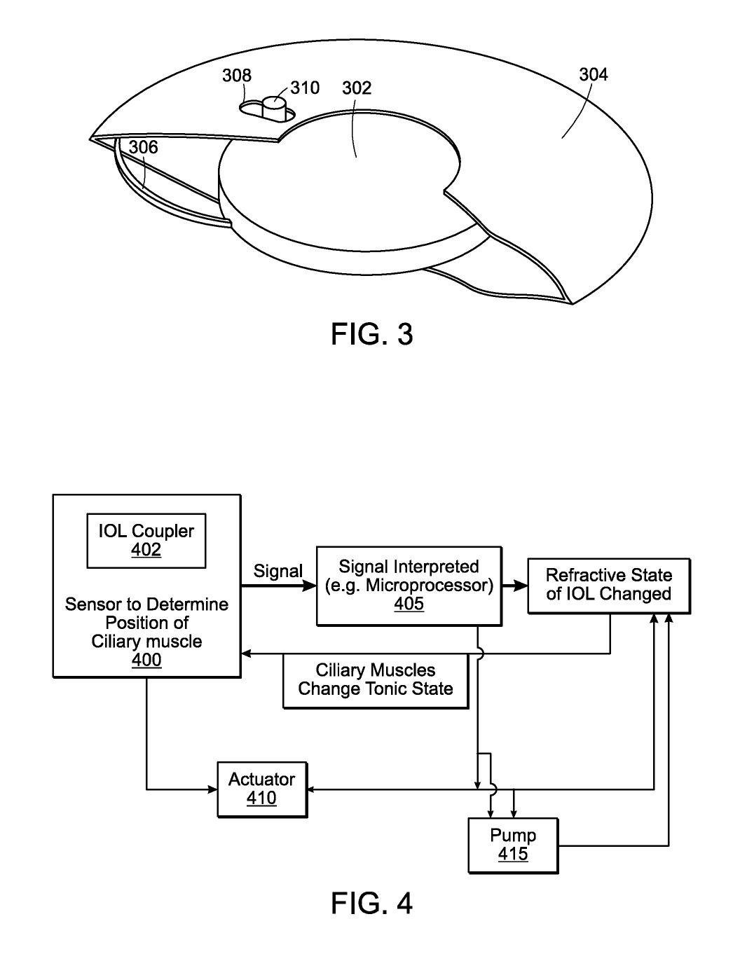

FIG. 3 shows an IOL 302 that does not change focal length with a shape change or positioning between multiple optic surfaces. Examples of these lenses include monofocal IOLs, multifocal IOLs, toric IOLs, astigmatism-reducing IOLs, aberration inducing/reducing IOLs, and position-shifting IOLs such as the crystalens. The coupling feature 310 is received within the coupling aperture 308 in the lens capsule 304. The coupling feature 310 ensures proper angular positioning of the IOL 302. Likewise, it can be used to maintain centering of the IOL 302. Here the coupling aperture 308 is shown as a slot, but this is for illustrative purposes; representative coupling apertures include slots, holes and slots, and other shapes that maintain contact as the lens capsule moves during accommodation.

In this embodiment, the IOL 302 includes a haptic feature 316—e.g., a plastic side strut that holds the IOL 302 in place within the capsular bag inside the eye. Coupling features may be attached to a haptic, fluid reservoir, fluid-filled haptic or actuation portion of the lens such as the hinge 214 shown in FIG. 2. As noted, the coupling features move with the lens capsule and can exert force causing the lens to accommodate, as in the case of accommodating IOLs. This can be actuated through a fluid-filled haptic, in which case the fluid travels from the haptic to the lens. In other embodiments, a fluid-filled reservoir shifts position, or the volume of fluid inside the reservoir changes, with movement of the lens capsule.

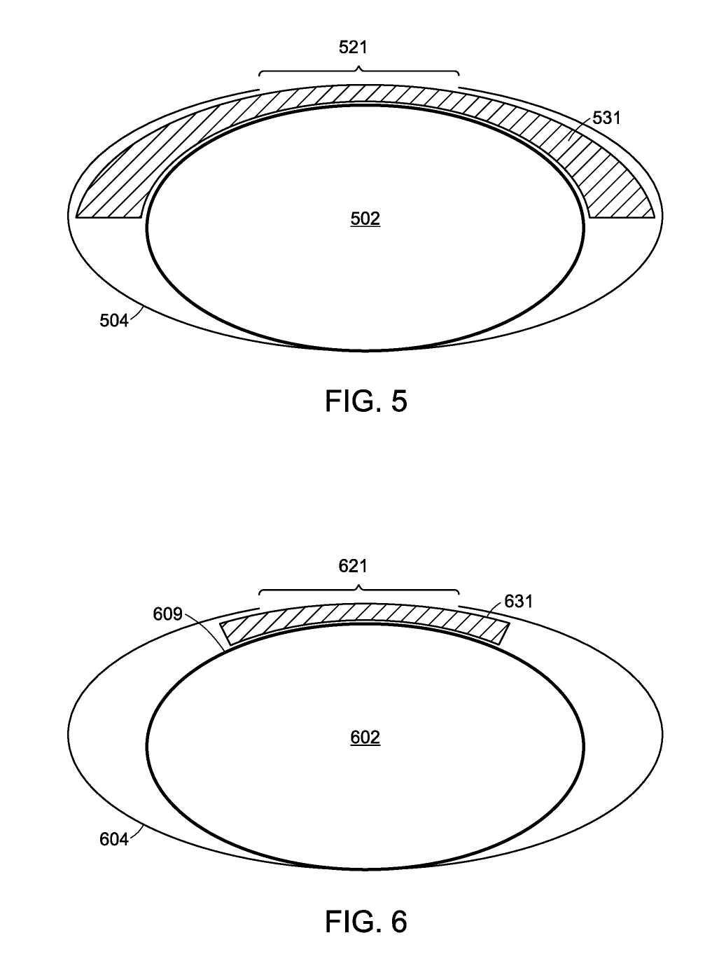

FIG. 5 depicts an embodiment in which an intermediate member is placed between the IOL 502 and the patient's lens capsule 504, providing support across any capsulorhexis opening 521 to prevent subluxation of the IOL 502 from either the anterior or posterior capsulorhexis. In particular, the intermediate member takes the form of a capsule coupler 531, which is implanted within the lens capsule 504 and wholly or partially encapsulates the IOL 502.

The capsule coupler 531 is a standalone component having opposed surface profiles contoured to complement, and interface smoothly with, the contour profiles of the lens capsule 504 and the IOL 502. The complementary profiles may simply be the curvatures of each surface to maximize surface area. The complementary profiles may additionally include one or more raised coupling features and coupling apertures. These coupling interfaces retain movement, migration, tilt, or rotation of the IOL 502 within the lens capsule 504.

In various embodiments, the capsule coupler 531 is designed to be easily manipulated with multiple degrees of freedom. Since it is not integrated into the IOL 502, during surgery it can be manipulated into the correct position and orientation without risk of damaging the IOL. (Damage is especially problematic for fluid-filled IOLs, as fluid leakage can adversely affect the IOL's optical properties.) Certain capsule couplers may have press-fit, latch, or other mechanical structures that secure the IOL in place and interface with (e.g., couple to) one or more IOL surfaces and/or IOL haptic surfaces.

In certain embodiments, the capsule coupler 531 is manufactured of the same material as the IOL to increase stiction and other static-interface force properties (e.g., if the outer surface of the IOL is silicone, the capsule coupler is also made of silicone). The surfaces of the capsule coupler may further be surface-treated (using, e.g., chemistry-altering surface treatments, etching, vapor deposition, etc.) to increase inter-surface interaction. Surface treatments are applied in select locations or are selected so as not to interfere with the optical properties of the IOL. In other embodiments, the surface properties of the capsule coupler are less adherent to the IOL, thereby allowing the IOL to move more freely with respect to the capsule coupler. Examples of this type of surface modification include parylene deposition into or on the polymer, e.g., parylene deposition into the silicone pores of a silicone capsule coupler.

FIG. 6 shows an embodiment in which changes in the anterior surface 609 of the IOL directly affect the capsule coupler 631. The capsule coupler is minimally larger than the capsulorhexis opening 621, and may be integrated with the IOL 602 prior to implantation. Alternatively, to minimize surgical incision size, the IOL 602 and capsule coupler 631 may be implanted in successive steps. For example, the IOL 602 and capsule coupler 631 may be folded and inserted into either a single or separate insertion tools. Upon insertion and ejection from the insertion tool(s), the IOL 602 and capsule coupler 631 naturally unfold into their conformal state. In the case of a fluid filled IOL, the IOL is also filled during insertion by a fluidic line incorporated in the insertion tool or accessed by another tool used in conjunction therewith. A separate manipulator is optionally used to move or maintain the capsule coupler before, during, and/or after IOL filling.

The minimally sized capsule coupler depicted in FIG. 6 ensures that the IOL 602 does not bulge through the capsulorhexis 621. For accommodating IOLs, it promotes the transmission of forces in an axial direction (i.e., forces from the anterior and posterior portion of the eye) to promote the shape change required for accommodation to occur in the optical zone of the IOL. In cases of good stiction between the accommodating IOL, capsule coupler, and lens capsule, any change in the lens capsule becomes a direct shape change in the anterior surface 609 of the IOL. By mechanically coupling the capsule coupler to the lens capsule, the lens capsule is allowed to apply not only force to the capsule cover, but also strain. In essence, in certain embodiments of the invention the capsule coupler functions to reinforce and reconstruct a portion of the natural lens capsule. The modulus of elasticity and thickness are preferably matched to the natural lens capsule properties. The matching is a cumulative outcome of ratios of thickness and modulus of elasticity (e.g., thinner with a higher modulus than the lens capsule, or thicker with a lower modulus than the lens capsule in order to accommodate the IOL to be inserted within.)

The capsule coupler may additionally contain features or be treated to limit fibrosis or cell migration. Proliferation and migration of epithelial cells from the peripheral posterior capsular bag is a common post-operative complication of cataract surgery that leads to anterior and/or posterior capsule opacification and decreased visual acuity. The capsule coupler may include one or more circular protrusions that cause an angular deformity of the surrounding lens capsule, thereby preventing migration of the lens epithelial cells. In other embodiments, a circular region of the capsule coupler adheres to the lens capsule, causing it to undergo an angular deformity with strain that prevents migration of lens epithelial cells. For example, the coupler may adhere to the capsule mechanically (e.g., with microfeatures such as grooves or circular angled edges that form an angular protrusion) or with an adhesive (which may be temperature-sensitive, e.g., poly(N-isopropylacrylamide or PNIPAM)). Alternatively or in addition, the capsule coupler may be coated with a therapeutic agent to prevent post-surgery complications, e.g., capsular fibrosis, inflammation, and capsular contraction.

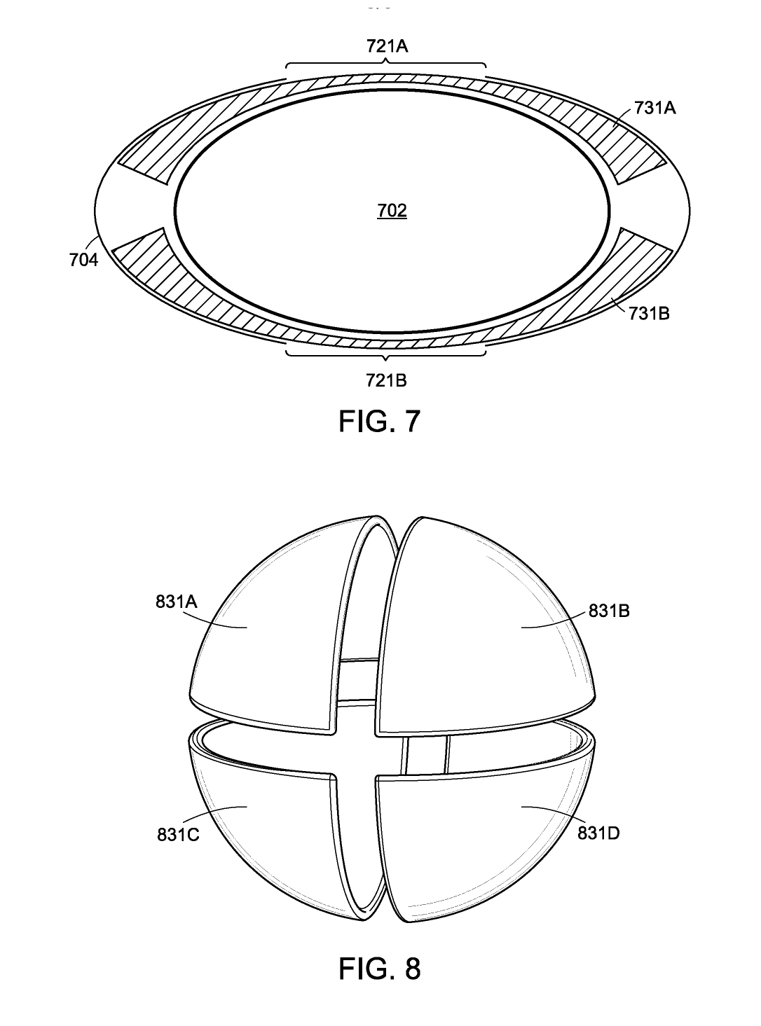

FIG. 7 shows a multi-section capsule coupler in two sections 731A, 731B, which extend over the anterior capsulorhexis 721A and posterior capsulorhexis 721B, respectively. The multi-section capsule coupler 731 further minimizes the surgical incision size as the capsule coupler may be implanted in sequential steps and each individual component folded into smaller configurations for implantation. The multi-section capsule coupler 731 may be implanted in different configurations including two or more components—e.g., a left and right portion, or quadrants 831A-831D as shown in FIG. 8, to transfer radial and axial forces from the lens capsule 704 to the IOL 702. Multi-section capsule couplers may additionally have breakable reinforcing members that connect two or more capsule coupler sections. These reinforcing members may be manipulated to adjust the tension on the lens capsule. When all reinforcing members are intact, there is greater tension on the lens capsule as there is more resistance to the forces applied by the lens capsule. Reinforcing members may be broken mechanically, optically or thermally, e.g., by UV exposure, Nd:YAG laser, femtosecond laser or other means. As more reinforcing members are broken, there is less resistance to the forces applied by the lens capsule and the lens capsule therefore experiences less tension, allowing for further lens capsule movement during accommodation.

In yet another embodiment, the capsule cover entirely or almost entirely encapsulates the IOL. The capsule cover contains one slit comparable in size to the incision required for IOL incision (e.g. 0.5 mm to 5 mm according to the IOL selected). The capsule cover is implanted in a collapsed form and regains its unfolded state by injection of gas or liquid through the slit or by mechanical manipulation. The IOL is then inserted through the corneal incision and through the slit in the capsule cover, and is then ejected from the insertion tool into the internal space of the capsule coupler. Upon removal of the insertion tool, the slit in the capsule cover closes, thereby almost entirely encapsulating the IOL within. The capsule cover slit is preferably created outside of the optical line of sight of the IOL to minimize any effect on the optical properties of the lens. These embodiments are analogous to reconstructing of the lens capsule. During implantation, large portions of the lens capsule may be removed without losing overall mechanical and structural support because the capsule cover supports the remaining capsule and connection to ciliary muscles and zonules. Minimizing ruptures in the lens capsule not only improves mechanical and structural support but also reduces the incidence and effect of inflammation, fibrosis, and posterior and anterior capsule opacification.

In the case of a fluid-filled IOL the capsule coupler may be pierced, preferably outside of the optical line of sight, by a filling system needle or cannula to access the IOL to adjust fill volume and contents. The capsule cover will reseal the hole once the filling needle is removed. By creating a large enough stiction surface, the capsule coupler may additionally function as a large self-sealing valve, thereby obviating the need for a separate valve in the IOL itself, and making the whole capsule coupler a large valve that may be accessed from any area and angle. In the case of an accommodating IOL, the capsule coupler is flexible and does not attenuate the force exerted by the lens capsule. Instead, the capsule coupler transfers forces (radial, axial, vertical, and horizontal) from the lens capsule to the IOL by maximizing the surface area between components to cause the IOL to accommodate.

By using coupling features between the IOL and the lens capsule, it is possible to add sensors to monitor, and actuators to alter, the state of the lens capsule. These may attach to the lens capsule in the same manner as, or along with, the IOL or capsule coupler. Sensors may provide feedback to indicate the position, focusing or accommodative state of the eye, or to otherwise monitor the status of the eye. For example, an IOL-coupled device may act as a mechanical feedback facility for a sensor. In one implementation, a tethered IOL-coupled device in the lens capsule is anchored to another portion of the lens capsule, or to the lens capsule itself. Motion of the IOL coupler provides mechanical feedback to a sensor, which responsively actuates the IOL—e.g., by providing an electrical actuation signal that alters the IOL power. The sensor may act directly on the IOL or via a microprocessor. The sensor may also provide diagnostic information to the clinician regarding the refractive state of the eye, the amount of accommodation that the eye is capable of, motion of the ciliary muscle/lens capsule, and/or pressure information regarding accommodation of the eye.

FIG. 4 illustrates a sensor 400 mechanically coupled to or integrated with an IOL for monitoring the status of the lens capsule. The sensor 400 receives information regarding the lens capsule by means of an IOL coupler 402, which is received within a special aperture in the lens capsule. The sensor communications with a signal-interpretation device 405, which also controls the state of the IOL, via an actuator 410, and is capable of delivering an actuator signal that causes the actuator 410 to change the the refractive state of the IOL. The actuator 410 may alter the conformation of the IOL directly, suitable piezo or MEMS components as are well-known in the art, by moving the coupling features so as to enhance or reduce the degree of ciliary contraction, or to move a portion of the IOL. In various embodiments, electrically actuated features can be integrated within the IOL or the capsule coupler itself, creating an electrically active lens and/or an electrically active capsule coupler that actuates an accommodative IOL; suitable actuators include electrowetting, electroactive polymers, temperature-actuated materials, electroelectrolysis, electro-osmosis. These alter the conformal shape of the IOL in response to an actuator signal. Alternatively or in addition, the actuator 410 (or the signal interpreter 405 directly) may operate a pump 415 that drives a fluid into or out of the IOL in order to change its lens power.

After the IOL changes refractive state, the refractive state of the eye further adjusts. The instantaneous refractive state of the eye is determined by the ciliary muscles as they change contraction state, which is detected, on a continuous basis, by the sensor 400 based on mechanical interaction between the coupler 402 and the lens capsule. For example, the sensor 400 may be or include a strain gauge that measures the strain on a coupling feature, which directly indicates the ciliary contractive state. The same information be obtained by a sensor that measures the positional change in the coupling feature. The force exerted by the ciliary muscle may be computed based on the observed strain and positional change of the coupling feature and its modulus. Other sensors that may be used include fluidic sensors and pressure transducers.

For example, the actuator 410 may be a force transducer that acts via the coupling features to assist the patient's ciliary muscle to achieve focus, at which point the ciliary muscle enters a state of tonus that may serve as a feedback signal to the signal interpreter 405 to cease force application. One or more force transducers may be incorporated to provide additive force transduction and various angles of force application. If, over time, the signal interpreter 405 determines that the degree of force necessary to achieve focus is increasing and will soon exceed the capability of the force transducer 410, the signal interpreter 405 may activate the pump 415 to drive fluid into the IOL and thereby increase its power.

The signal interpreter 405 may be realized as a separate console implementing general-purpose computer, or may instead be an embedded microcontroller. In either case, the signal interpreter 405 may include a suitable processing unit, a system memory, and a system bus that couples various system components including the system memory, the processing unit, and input/output ports that interface to the sensor 400, the actuator 410, and the pump 415. Computers typically include a variety of computer-readable media that can form part of the system memory and be read by the processing unit. By way of example, and not limitation, computer readable media may comprise computer storage media and communication media. The system memory may include computer storage media in the form of volatile and/or nonvolatile memory such as read only memory (ROM) and random access memory (RAM). A basic input/output system (BIOS), containing the basic routines that help to transfer information between elements, such as during start-up, is typically stored in ROM. RAM typically contains data and/or program modules that are immediately accessible to and/or presently being operated on by processing unit. The data or program modules may include an operating system, application programs, other program modules, and program data. The operating system may be or include a variety of operating systems such as Microsoft WINDOWS operating system, the Unix operating system, the Linux operating system, the Xenix operating system, the IBM AIX operating system, the Hewlett Packard UX operating system, the Novell NETWARE operating system, the Sun Microsystems SOLARIS operating system, the OS/2 operating system, the BeOS operating system, the MACINTOSH operating system, the APACHE operating system, an OPENSTEP operating system or another operating system of platform.

A stand-alone console may also include other removable/nonremovable, volatile/nonvolatile computer storage media. For example, a hard disk drive may read or write to nonremovable, nonvolatile magnetic media. A magnetic disk drive may read from or writes to a removable, nonvolatile magnetic disk, and an optical disk drive may read from or write to a removable, nonvolatile optical disk such as a CD-ROM or other optical media. Other removable/nonremovable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The storage media are typically connected to the system bus through a removable or non-removable memory interface.

The processing unit that executes commands and instructions may be a general purpose computer, but may utilize any of a wide variety of other technologies including a CSIC (customer-specific integrated circuit), an ASIC (application-specific integrated circuit), a logic circuit, a digital signal processor, a programmable logic device such as an FPGA (field-programmable gate array), PLD (programmable logic device), PLA (programmable logic array), RFID processor, smart chip, or any other device or arrangement of devices that is capable of implementing the steps of the processes of the invention.

Having described certain embodiments of the invention, it will be apparent to those of ordinary skill in the art that other embodiments incorporating the concepts disclosed herein may be used without departing from the spirit and scope of the invention. For example, various features described with respect to one particular device type and configuration may be implemented in other types of devices and alternative device configurations as well. Accordingly, the described embodiments are to be considered in all respects as only illustrative and not restrictive.

What is claimed is:

1. An intraocular lens comprising:
a polymeric lens body having a refractive power and configured for implantation to a patient's lens capsule; and
one or more coupling features on the lens body for anchoring the lens body to the lens capsule.

2. The lens of claim 1, wherein the one or more coupling features include a plurality of protrusions from a surface of the lens body.

3. The lens of claim 2, wherein the protrustions each include a catch that latches over the lens capsule.

4. The lens of claim 3, wherein the catch is a hook, ledge, circular protrusion, or coned feature.

5. The lens of claim 1, wherein the one or more coupling features include one or more adhesive patches that adhere the lens body to the lens capsule.

6. The lens of claim 5, wherein the said one or more adhesive patches include a chemical adhesive.

7. The lens of claim 5, wherein the said one or more adhesive patches include a mechanically featured adhesive.

8. The lens of claim 1, wherein in the one or more coupling features is a surface treatment on the lens body promoting adhesion to the lens capsule.

9. The lens of claim 1, wherein the one or more coupling features are outside a central optical axis of the lens.

10. The lens of claim 9, wherein the one or more coupling features are outside a central 2 mm of the lens.

11. The lens of claim 9, wherein the one or more coupling features are outside a central 3 mm of the lens.

12. The lens of claim 1, further comprising an intermediate member with coupling features complementary to the lens body and the lens capsule.

13. The lens of claim 12, wherein the intermediate member comprises a plurality of segments.

14. The lens of claim 13, wherein the segments are breakably joined to each other.

15. A sensor coupling system comprising:
a polymeric lens body having a refractive power and configured for implantation to a patient's lens capsule;
one or more coupling features on the lens body for anchoring the lens body to the lens capsule;
a sensor for measuring a force exerted on the one or more coupling features;
a signal interpreter for interpreting sensor measurements; and
means, responsive to the signal interpreter, for altering the refractive power of the lens body based at least in part on measurements from the sensor.

16. The system of claim 15, wherein the signal interpreter is configured to determine a state of the patient's eye.

17. The system of claim 15, wherein the means responsive to the signal interpreter comprises an actuator for altering a conformation of the lens body via the coupling features.

18. The system of claim 15, wherein the means responsive to the signal interpreter comprises a pump for driving a fluid into and out of the lens body.
